(19) United States
(12) Patent Application Publication
(10) Pub. No.: US 2009/0186110 A1
(43) Pub. Date: Jul. 23, 2009

(54) GUAVA EXTRACT

(76) Inventor: Thomas Eidenberger, Steyr (AT)

Correspondence Address: FULBRIGHT & JAWORSKI L.L.P.;Attn: MN IP Docket, 600 Congress Avenue, Suite 2400, Austin, TX, 78701, US

(21) Appl. No.: 12/093,264

(22) PCT Filed: Nov. 8, 2006
(86) PCT No.: PCT/AT2006/000454

§ 371 Date: Dec. 11, 2008

(30) Foreign Application Priority Data
Nov. 9, 2005 (AT) A 1833/2005

(51) Int. Cl. A61K 36/61; A61P 3/04; A61P 3/10; C07D 311/30

(52) U.S. Cl. 424/774; 424/777; 424/775; 549/400

(57) ABSTRACT

The present invention relates to the use of an extract of a plant selected from the group consisting of Psidium cattleianum, Psidium cattleianum ssp. Lucidum, Psidium guajava, Psidium guineense, Psidium littorale, Psidium molle and Psidium schiedeanum for the manufacture of a medicament for the treatment of a disease and/or condition related with and/or caused by activity of DP-IV or DP-IV like enzymes.

The present invention relates to the pharmaceutical use of an extract of a plant selected from the group consisting of Psidium cattleianum, Psidium cattleianum ssp. Lucidum, Psidium guajava, Psidium guineense, Psidium littorale, Psidium molle and Psidium schiedeanum.

In recent years, it was found that some diseases associated with hyperglycemia, such as especially diabetes mellitus I and II, are related to the activity of the dipeptidyl peptidase IV (DP-IV) enyzme.

DP-IV is a membrane-associated peptidase of 766 amino acids that is widely distributed in numerous tissues. DP-IV also exists as a soluble circulating form in plasma. Significant DP-IV activity is detectable in plasma from humans and rodents. The first biological principle of membrane-associated DP-IV relates to intracellular signalling pathways. The second principal biological activity of DP-IV is its enzymatic function in plasma. DP-IV prefers as peptidase substrates with an amino-terminal proline or alanine at position 2, but may also cleave substrates with non-preferred amino acids at position 2. Observations from a number of laboratories delineated the importance of DP-IV-mediated inactivation of GLP-1 as a key determinant of GLP-1 bioactivity. Several DP-IV inhibitors have been characterized, and they appear to lower blood glucose in diabetic rodents via prolongation of GLP-1 and GIP action in plasma. The use of DP-IV inhibitors for the treatment of diseases such as diabetes mellitus has, for example, been proposed in U.S. Pat. No. 6,500,804 B2.

It has now surprisingly been found that guaijaverin, a member of the chemical class of Flavonols, is very effective as a DP-IV inhibitor, therefore rendering this compound suitable for the treatment of diseases associated with DP-IV activity.

Guaijaverin has the formula I

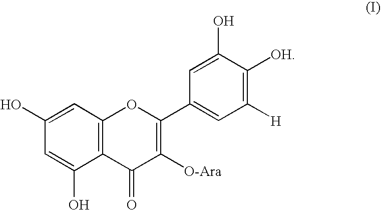

Compounds of the flavonol group are reported to have an inhibiting effect on aldose reductase (cf. Matsuda et al., Pure Appl. Chem. Vol. 74 (2002), No. 7, pp. 1301-1308; Chaudry et al. Biochem Pharmacol. 1983, 32 (13), 1995-1998; Yoshikawa et al., Chem. Pharm. Bull (Tokyo) 1998, 46 (1), 113-119). Compounds of this class, therefore, have been proposed (especially in terms of herbal plants medicine, cf. www.rain-tree.com/pedra.htm or http://www.e2121.com/ffood.html) for the treatment of conditions such as retinopathic conditions caused by diabetes.

Furthermore, U.S. 2002/0147353 A1, WO 2003/026561 A2, WO 2001/049285, JP 09-094077A and JP 2000-001435 A disclose compounds of the flavonol group and their effects.

However, it was only now discovered that guaijaverin is effective against DP-IV activity and, hence, suitable for the treatment of diabetes or related diseases per se.

Guaijaverin is available by preparing extracts from plants containing guaijaverin, such as Myrcia multiflora, and isolating the desired compounds from said extract by methods known as such.

One aspect of the present invention relates to the use of an extract of a plant selected from the group consisting of Psidium cattleianum, Psidium cattleianum ssp. Lucidum, Psidium guajava, Psidium guineense, Psidium littorale, Psidium molle and Psidium schiedeanum for the manufacture of a medicament for the treatment of a disease and/or condition related with and/or caused by activity of DP-IV or DP-IV like enzymes. A preferred plant of this group is Psidium guajava. Psidium guajava is a plant of the Myrtoideae family.

The plants of the above listed group, especially Psidium guajava, contain significant amounts of guaijaverin.

The plant extract used according to the present invention may be an extract of the leaves, the fruits and/or of the bark of the plant.

The extract may be prepared with a solvent selected from the group of water, methanol, ethanol, acetone, ethyl acetate and mixtures thereof, by methods known as such. Furthermore, the extract may be prepared by alternative methods, such as membrane filtration techniques using e.g. the juice of the fruit of the plant.

Preferably, the extract used according to the invention is present in a solid form, such as a powder.

The extract used according to the invention may preferably contain an amount of guaijaverin of 0.5% by weight or more, preferably 10 to 50% by weight. Typically, the extract may contain an amount of guaijaverin of 2% by weight to 4% by weight.

A further aspect of the present invention relates to the use of guaijaverin and/or a pharmaceutically acceptable salt or ester thereof for the manufacture of a medicament for the treatment of a disease or condition related with or caused by activity of DP-IV or DP-IV-like enzymes.

The use of guaijaverin and/or of the plant extract used according to the invention is especially suitable for the treatment of a disease and/or condition which is a glucose metabolism disorder, such as diabetes mellitus, obesity and/or atherosclerosis.

Furthermore, guaijaverin and/or the plant extract used according to the invention may be used for other therapeutic purposes, such as for lowering LDL cholesterol, as an antioxidant, as an analgesic agent, and as a haemostatic agent, e.g. for relieving conditions associated with women's menstruation.

A further aspect of the present invention relates to the extract of a plant selected from the group consisting of Psidium cattleianum, Psidium cattleianum ssp. Lucidum, Psidium guajava, Psidium guineense, Psidium littorale, Psidium molle and Psidium schiedeanum for use as a medicament.

Guaijaverin and/or the plant extract used according to the invention may be converted into pharmaceutically acceptable compositions, using pharmaceutically acceptable excipients, by methods known as such in the art. Administration may be carried out in various manners known as such, e.g. orally, topically, or as an injection.

Preferably, the content of guaijaverin is 0.5% by weight or more in such compositions.

Guaijaverin and/or a pharmaceutically acceptable salt or ester thereof, and/or of an extract of a plant selected from the group consisting of Psidium cattleianum, Psidium cattleianum ssp. Lucidum, Psidium guajava, Psidium guineense, Psidium littorale, Psidium molle and Psidium schiedeanum, may also be used in the form of a nutritional ingredient, for example as a component of functional food, in a soft drink, or as a component in a cosmetic product.

Furthermore, guaijaverin and/or the plant extract used according to the invention may be mixed with other plant extracts like e.g extracts from bitter melon, mulberry leaves, and banaba leaves.

In the following, the invention will be described in more detail on the basis of the figures and examples disclosing preferred embodiments of the invention:

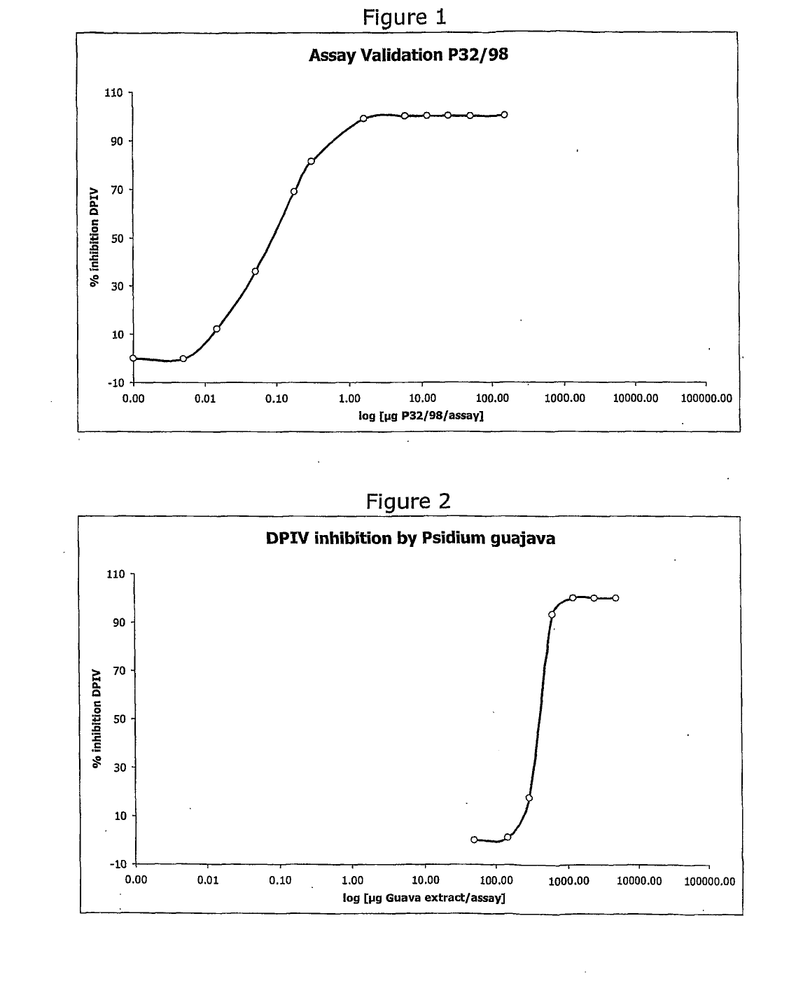

FIG. 1 shows the inhibition of DP-IV by the synthetic inhibitor P32/98.

FIG. 2 shows the inhibition of DP-IV by an ethanolic extract of Psidium guajava.

### EXAMPLE 1

### General Description of the Preparation of an Ethanolic Extract from Psidium guajava

The production process of preparing an extract from the leaves of Psidium guajava is described by following process steps:
Pulverize Psidium guajaya's leaves to fine powder
Extract with ethanol.
Reduce the pressure of extract liquid and concentrate until evaporation of ethanol.
Eliminate insoluble impurities by centrifugation and obtain a clarified liquid.
Load the clarified liquid onto a chromatographic column filled with a macroporous resin, clean off further impurities by water and then desorb by ethanol and collect the ethanolic eluate.
Dry the eluate to yield Psidium guajava extract.

1. Extract: Powdered Psidium guajava leaves are extracted two times with 80% ethanol at 60±2° C. . Extraction time is 2 hours each. The ratio of the final ethanol volume to the raw material powder weight is 8 to 1.

2. Concentration: The pressure is reduced, and the extract liquid is concentrated at 60±2° C. until there is no ethanol left. The vacuum should be better than −0.08 MPa.

3. Chromatography: The clarified liquid obtained from the centrifuge is loaded onto a macroporous resin. Part of the impurities are cleaned with water. Elution is carried out with 95% ethanol until the liquid is clarified and the color is a little yellow. The velocity of flow should be controlled at 15-20 mL/min. The part of ethanol desorbed liquid is collected.

4. Drying: The pressure is reduced, and the eluate is concentrated/dried. The vacuum should be better than −0.08 MPa. A final powder is obtained.

### EXAMPLE 2

### Production of Psidium guajava Extract

500 g pulverized raw material (fresh Psidium guajava fruit) are weighed and transferred into 2000 mL 80% ethanol (3-neck boiling flask). The mixture is mixed gently for 2 hours at 60° C. The solution is filtered. The filtrate is collected, and extraction is repeated with 2000 mL 80% ethanol under the same conditions. The solution is again filtered, and both filtrates are combined.

The filtrate is concentrated under reduced pressure at 60° C. until no ethanol is left. The vacuum degree is −0.09 MPa. The resulting ethanol-free liquid is centrifuged to remove solid particles. 200 mL water are added to the pellet, and the mixture is again centrifuged. Both supernatants are combined. The clarified liquid is loaded onto a macroporous resin (Type Amberlite XAD4) and rinsed first with 800 mL water at a flow rate of 17 mL/min to wash off part of the impurities. Then it is switched to 1000 mL 95% ethanol at a flow rate of 8.5 mL/min for desorption, and the eluate is collected for 2 hours. The eluate is concentrated at 60° C. under reduced the pressure, followed by drying for 5 hours.

1.4 g Psidium guajava extract is obtained. The content of quercetin is determined to be 0.10%, and the content of guaijaverin is determined to be 0.82%.

### EXAMPLE 3

### Production of Psidium guajava Extract

20 kg pulverized Psidium guajava's leaves powder are weighed and put into a 250 L boiler, then 160 L 80% ethanol are added, and the mixture is mixed for 2 hours at 60° C. The solution is filtered, and again 160 L 80% ethanol are poured into the residue. The mixture is extracted further for 2 hours at 60° C., filtered, and then the filtrate liquids of the two extraction steps are mixed together. The resulting mixture is concentrated under reduced pressure at 60° C. until there is barely any ethanol left. The vacuum degree is −0.09 MPa. The obtained mixture is centrifuged. 40 L of water are then used to wash the residue after centrifugation, and the filtrate liquids are mixed together. The combined filtrates are separated through a macrocrosslinked macroporous resin (Type Amberlite XAD4). First, 160 L water are used to wash the resin after absorption, by which part of the impurities can be eliminated. Then, 350 L 80% ethanol are used for the desorption step. The yellow-brown desorbed liquid is collected. The desorbed liquid is concentrated under reduced pressure at 60° C. Afterwards it is dried in a vacuum dryer for 5 hours.

1.8 kg Psidium guajava extract is obtained. The content of guaijaverin is 12.44% with 1.02% quercetin as determined by HPLC analysis.

The above product can be further concentrated. The product is dissolved in 200 L water. The solution is separated through a polyamide resin (a polyamide 6 resin from Messrs. Sorbent Technologies, Inc.): First, 40 L water are used to wash the resin to remove part of the impurities. Then, 60 L 80% ethanol are used for the desorption step, and the yellow-brown desorbed liquid is collected. The desorbed liquid is concentrated under reduced pressure at 60° C., then it is dried in a vacuum dryer for 5 hours.

0.45 kg Psidium guajava extract is obtained. The content of guaijaverin is 42.09% with 0.96% quercetin as determined by HPLC analysis.

### EXAMPLE 4

### Investigation of the DP-IV Inhibiting Effect of a Psidium guajava Extract

### Methods:

DP-IV activity was measured by a colorimetric assay.

Gly-Pro-4-NA (G0513, Sigma, St. Louis, Mo.), a (synthetic) chromogenic substrate of DP-IV, is hydrolyzed by DP-IV into the dipeptide glycine-proline and 4-nitroaniline, whose rate of appearance was followed quantitatively at 405 nm.

400 μL assay buffer (9.5 g HEPES/l distilled water, pH adjusted to 7.0, H4034, Sigma, St. Louis, Mo.), 150 μL human plasma and 100 μL inhibitor solution (or solvent) were transferred into a photometer cuvette, gently mixed and pre-incubated at 37° C. for 3 minutes. The assay is then started by addition of 70 μL substrate solution (8.6 mg Gly-Pro-4-NA in 10 mL assay buffer). The increase of absorption at 405 nm was recorded over a period of 20 min.

DP-IV activity is expressed as the linear change in optical density over 20 min (Δ Abs/min).

### Sample Preparation:

A Psidium gujava extract obtained according to example 3 was extracted at 45° C. for 24 hours in distilled water under stirring conditions. Thereafter the extract was cleared by centrifugation (15,000 rpm, 15 min.), filtration (syringe filter, 0.45 μM), appropriately diluted and submitted to the test assay.

The concentration of the extract was 5 g powder/100 mL water. Dilutions were prepared from the cleared extract by addition of water.

For comparison purposes, DP-IV was inhibited by P32/98 (3N-[(2S,3S)-2-amino-3-methyl-pentanoyl]-1,3-thiazolidine hemifumarate), a synthetic enzyme inhibitor.

A stock solution of 1.60 mg P32/98/mL assay buffer was prepared and diluted with assay buffer to yield concentrations between 0.50 mg/mL and 0.05 mg/mL. 100 μL of these solutions were added to the assay as “inhibitor” solution. Comparison experiments were carried out in the same test assay system as described further below in the “Results” section.

### Data Evaluation:

Results are expressed as %-inhibition derived from the comparison of test results obtained in samples with no inhibitor added to results obtained in samples with added inhibitors or Psidium guajava extract (both in different concentrations).

No inhibition (0%) in a test sample indicates the same increase in absorption compared with a sample with no inhibitor added. Full inhibition (100%) indicates no apparent increase in absorption.

All test results represent the average of 2 samples. The relative standard deviation of these replicate samples was always less than 7%.

### Results:

The assay was calibrated using well known routine procedures:

The pH-optimum of the test assay system was shown to be in the range between pH=6.0 and pH=8.0. An assay temperature range between 32 and 42° C. does not significantly affect the enzyme activity. Any substrate concentration between 5 and 10 μg/10 mL yielded maximum enzyme activity. At the substrate concentration chosen, the increase in absorption was. shown to be linear up to 45 minutes. Under the assay conditions chosen, plasma volumes between 100 and 200 μL were shown to yield a dose-dependent, parallel shift of the increase in absorption.

Replicate tests under the final test assay conditions yield a relative standard deviation of less than 7%.

Inhibitions of the enzyme by well known unspecific enzyme inhibitors and various solvents yielded results that are shown in Table 1.

TABLE 1
Effect of unspecific enzyme inhibitor on DP-IV activity

| Enzyme Inhibitors (100 mM) | %-Inhibition DP-IV |
|---|---|
| Ethylendiamine•HCl | 19.0 |
| EDTA | 24.0 |
| Thimerosal | <1.0 |
| Methimazol | 10.0 |
| Mercaptoethanol | 15.3 |
| Zinc⁺⁺ | 19.3 |
| Ethanol | 52.8 |
| Methanol | 59.7 |
| DMSO | 75.3 |

As seen, the enzyme DP-IV is not substantially blocked by the unspecific enzyme inhibitors chosen. Mentionable inhibition was achieved by organic solvents. Due to these results, the extracts at hand were dissolved in water, as organic solvents were shown to block the enzyme activity, hence introduction of those solvents would have led to uninterpretable results.

The results of the tests carried out with synthetic inhibitor P 32/98 and Psidium guajava extract are shown in FIGS. 1 and 2, with the concentration of the respective inhibitor plotted on the abscissa, and the respective observed inhibition of DP-IV plotted on the ordinate.

As shown in FIG. 1, the synthetic inhibitor P32/98 yields a smooth dose/response inhibition curve. In the test assay system chosen, a concentration of approximately 0.10 μg/assay volume yielded a DP-IV inhibition of around 50%.

As shown in FIG. 2, the extract of Psidium guajava also yields a smooth dose/response inhibition curve. In the test assay system chosen, a concentration between 100-1.000 μg/assay volume yielded a DP-IV inhibition of around 50%.

Hence, Psidium guajava extract was shown to inhibit DP-IV substantially. The difference in potency between Psidium guajava and the synthetic inhibitor P 32/98 amounts to approximately 1.000.

### EXAMPLE 5

### Investigation of the DP-IV Inhibiting Effect of Guaijaverin

Unless indicated otherwise in the following, the materials and methods applied were the same as those of example 4.

### Sample Preparation:

Guaijaverin was dissolved in HEPES buffer (20 min. ultrasonication followed by shaking for 2 hours at room temperature), appropriately diluted and submitted to the test assay. Dilutions were prepared by addition of HEPES buffer. The concentrations tested were between 70-280 μg/mL test assay.

### Data Evaluation:

Results are expressed as %-activity derived from the comparison of test results obtained in positive control samples (no inhibitor added) to results obtained in samples with added guaijaverin at different concentrations.

100% activity in a test sample indicates the same increase in absorption compared in a sample with no inhibitor. Zero activity (0%) indicates no apparent increase in absorption.

All test results represent the average of 2 samples. The relative standard deviation of these replicate samples was always less than 5%.

### Results:

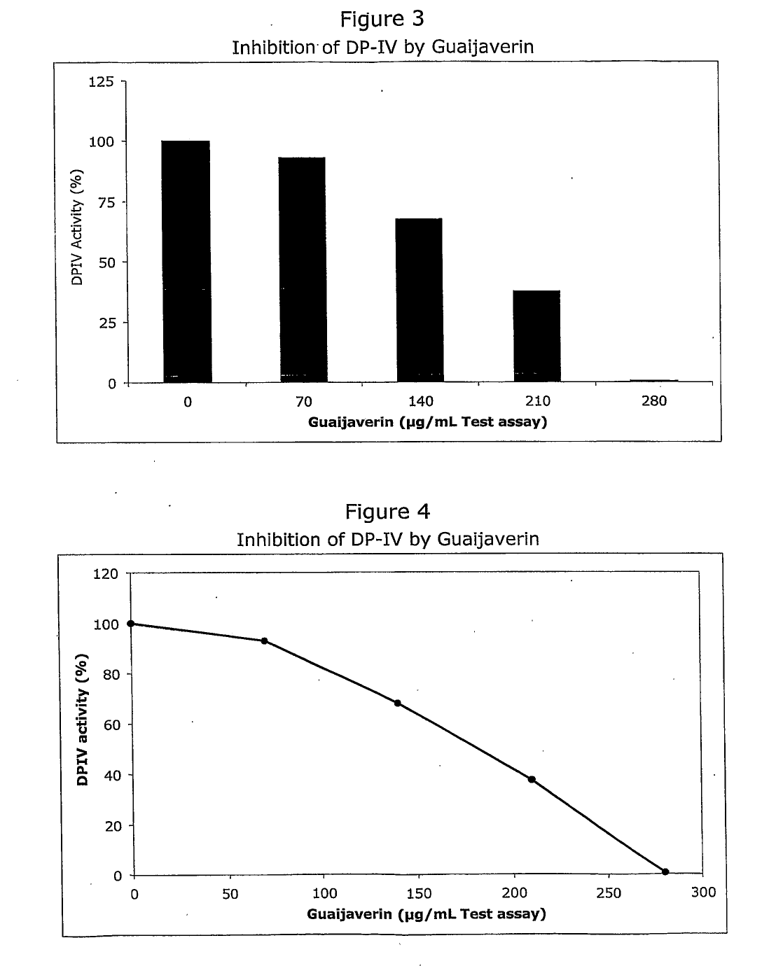

As shown in FIGS. 3 and 4, guaijaverin yields a clear, dose dependent inhibition of DP-IV. In the test assay system chosen, a concentration between 140-210 μg/mL test assay (100-150 μg/assay volume) yielded a DP-IV inhibition of around 50%.

1. Use of an extract of a plant selected from the group consisting of Psidium cattleianum, Psidium cattleianum ssp. Lucidum, Psidium guajava, Psidium guineense, Psidium littorale, Psidium molle and Psidium schiedeanum for the manufacture of a medicament for the treatment of a disease and/or condition related with and/or caused by activity of DP-IV or DP-IV like enzymes.

2. Use according to claim 1, wherein the plant is Psidium guajava.

3. Use according to claim 1 or 2, wherein the extract is an extract of the leaves, the fruits and/or of the bark of the plant.

4. Use according to any one of claims 1 to 3, wherein the extract is an extract prepared with a solvent selected from the group of water, methanol, ethanol, acetone, ethyl acetate and mixtures thereof.

5. Use according to any one of claims 1 to 4, wherein the extract is in a solid form.

6. Use according to any one of claims 1 to 5, characterized in that it contains an amount of guaijaverin of 0.5% by weight or more, preferably 10 to 50% by weight.

7. Use of guaijaverin and/or a pharmaceutically acceptable salt or ester thereof for the manufacture of a medicament for the treatment of a disease or condition related with or caused by activity of DP-IV or DP-IV like enzymes.

8. Use according to any one of the preceding claims, wherein the disease and/or condition is a glucose metabolism disorder, such as diabetes mellitus, obesity and/or atherosclerosis.

9. An extract of a plant selected from the group consisting of Psidium cattleianum, Psidium cattleianum ssp. Lucidum, Psidium guajava, Psidium guineense, Psidium littorale, Psidium molle and Psidium schiedeanum for use as a medicament.

10. Use of guaijaverin and/or a pharmaceutically acceptable salt or ester thereof, and/or of an extract of a plant selected from the group consisting of Psidium cattleianum, Psidium cattleianum ssp. Lucidum, Psidium guajava, Psidium guineense, Psidium littorale, Psidium molle and Psidium schiedeanum, as a nutritional ingredient, for example as a component of functional food, in a soft drink, or as a component in a cosmetic product.
